SEQUENCE LISTING (4 sequences)

SEQ ID NO: 1 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 2 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLTLGVLSLLLLVTGAFGFHLWRRQWRPRRFSALEQGIHPPQAQSKIEELEQEPEPEPEPEPEPEPEPEPEQL

SEQ ID NO: 3 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 4 - protein, 458 aa, Homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

(19) United States
(12) Patent Application Publication
(10) Pub. No.: US 2015/0132744 A1
(43) Pub. Date: May 14, 2015

(54) METHODS OF DETECTING CELLS LATENTLY INFECTED WITH HIV

(71) Applicant: Oregon Health and Science University, Portland, OR (US)

(72) Inventors: Remi FROMENTIN, Port St. Lucie, FL (US); Nicolas Chomont, Port St. Lucie, FL (US); Rafick-Pierre Sekaly, Cleveland, OH (US)

(73) Assignee: Oregon Health and Science University, Portland, OR (US)

(21) Appl. No.: 14/540,722

(22) Filed: Nov. 13, 2014

Related U.S. Application Data
(60) Provisional application No. 61/903,799, filed on Nov. 13, 2013.

(51) Int. Cl. G01N 33/569; G01N 33/50

(52) U.S. Cl. CPC G01N 33/56988; G01N 33/56972; G01N 33/505; G01N 2500/10
USPC 435/5

(57) ABSTRACT

Disclosed are methods of detecting and isolating CD4⁺ T cells latently infected with HIV as well as methods of screening for inhibitors of CD4⁺ T cells latently infected with HIV.

### CROSS-REFERENCE TO RELATED APPLICATIONS

This application claims priority to U.S. Provisional Application No. 61/903,799, filed Nov. 13, 2013, the entirety of which is incorporated by reference herein.

### ACKNOWLEDGEMENT OF GOVERNMENT SUPPORT

This invention was made with United States government support under the terms of grant number U19 AI096109, awarded by the National Institutes of Health. The United States government has certain rights in this invention.

### BACKGROUND

Antiretroviral therapy (ART) reduces HIV replication but does not cure HIV. The persistence of HIV in a small pool of long-lived latently infected resting CD4 T cells is a major barrier to viral eradication. Methods of identifying cells in which HIV persists are clearly necessary.

### SUMMARY

Several immune checkpoint blockers (ICBs) have been shown to actively reduce T cell activation, proliferation and cytokine production in CD4⁺ T cells, thereby acting as negative regulators of T-cell activation. In particular, PD-1, LAG-3 and TIGIT have been identified as markers associated with incomplete CD4 T cell restoration and HIV persistence during ART. Disclosed herein are methods involving the surprising discovery relating to identifying a cell comprising a latent HIV nucleic acid by detecting one or more of PD-1, LAG-3 and TIGIT, alone or in combination with each other or any other marker.

It is an object of the invention to efficiently identify cells latently infected with HIV.

It is an object of the invention to allow efficient sorting of cells latently infected with HIV.

It is an object of the invention to provide a population of cells that can be used to screen test compounds for potential therapeutics that inhibit (or eliminate) cells latently infected with HIV.

The methods involve obtaining a biological sample from a subject, the biological sample comprising CD4⁺ T cells. The sample is contacted with a labeled antibody that is conjugated to the label. The labeled antibody is specific for PD-1, AG-3, or TIGIT. The label can be detected on the surface of a CD4⁺ T cell that has bound the labeled antibody, thereby indicating that the CD4⁺ T cell expresses PD-1, LAG-3, and/or TIGIT on its surface. Detection of the label indicates that the cell comprises latent HIV nucleic acid.

### BRIEF DESCRIPTION OF THE DRAWINGS

FIG. 1 is a plot of the expression of the indicated immune checkpoint blockers PD-1, LAG-3, TIGIT, CTLA-4, BTLA, CD160, 2B4, and TIM-3 on CD4⁺ T cells isolated from HIV infected subjects undergoing antiretroviral therapy.

FIG. 2 shows the association between CD4 count and the frequency of CD4 T cells expressing PD-1, LAG-3, and TIGIT. Panel A shows a plot of total CD4 T cell count against the percentage of PD-1⁺ T cells isolated from HIV infected subjects undergoing antiretroviral therapy. Panel B shows a plot of total CD4 T cell count against the percentage of LAG-3⁺ T cells isolated from HIV infected subjects undergoing antiretroviral therapy. Panel C shows a plot of total CD4 T cell count against the percentage of TIGIT⁺ T cells isolated from HIV infected subjects undergoing antiretroviral therapy.

FIG. 3 is a plot showing copy number of integrated HIV DNA, total HIV DNA, 2-LTR HIV DNA and unspliced HIV RNA from HIV infected subjects undergoing antiretroviral therapy.

FIG. 4 shows that the frequency of CD4 T cells harboring intergrated HIV DNA was positively correlated with the expression of PD-1, LAG-3, and TIGIT. Panel A shows a plot of PD-1⁺; CD4⁺ T cells against integrated HIV DNA copy number per 10⁶ CD4 T cells from HIV infected subjects undergoing antiretroviral therapy. Panel B shows a plot of LAG-3⁺; CD4⁺ T cells against integrated HIV DNA copy number per 10⁶ CD4 T cells from HIV infected subjects undergoing antiretroviral therapy, Panel C shows a plot of TIGIT⁺ CD4⁺ T cells against integrated HIV DNA copy number per 10⁶ CD4 T cells from HIV infected subjects undergoing antiretroviral therapy.

### DETAILED DESCRIPTION

### Definitions

For the purpose of the current disclosure, the following definitions shall, in their entireties, be used to define technical terms, and to define the scope of the composition of matter for which protection is sought in the claims.

Antibody:

A polypeptide including at least a light chain or heavy chain immunoglobulin variable region which specifically recognizes and binds an epitope of an antigen or a fragment thereof, Antibodies are composed of a heavy and a light chain, each of which has a variable region, termed the variable heavy (VH) region and the variable light (VL) region. Together, the VH region and the VL region are responsible for binding the antigen recognized by the antibody.

The term “antibody” encompasses intact immunoglobulins, as well the variants and portions thereof, such as Fab fragments, Fab′ fragments, F(ab)′2 fragments, single chain Fv proteins (“scFv”), and disulfide stabilized RI proteins (“dsFv”). In some aspects, the antibody can be a camelid-derived antibody. In some aspects, the antibody can be an antibody derived from cartilaginous fishes. A scFv protein is a fusion protein in which a light chain variable region of an immunoglobulin and a heavy chain variable region of an immunoglobulin are bound by a linker. In dsFvs the chains have been mutated to introduce a disulfide bond to stabilize the association of the chains. The term also includes genetically engineered forms such as chimeric antibodies, and heteroconjugate antibodies (such as, bispecific antibodies). See also, Pierce Catalog and Handbook, Pierce Chemical Co., Rockford, Ill., 1994-1995; Kuby, J., Immunology, 3rd Ed., W.H. Freeman & Co., New York, 1997. The term includes both monoclonal and polyclonal types of antibodies, One or more monoclonal antibody species may be combined. An antibody of the present invention may be recombinant or produced using hybridoma technology.

The PD-1, LAG-3 and TIGIT binding antibodies can be full-length. In some aspects, the full-length antibodies can be selected from one of the following antibody types: IgG (as non-limiting examples: an IgG1, IgG2, IgG3, IgG4), IgM, and IgA (as non-limiting examples: IgA1, or IgA2), IgO, and IgE). In some aspects, the PD-1, LAG-3 and TIGIT binding antibodies can comprise an antigen-binding fragment (as non-limiting examples: a Fab, Fab′, F(ab′h or scFv fragment). In some embodiments, the antigen-binding fragment does not need to include an Fe domain or a CH2, CH3, or CH4 sequence. The antibody can include two heavy chain immunoglobulins and two light chain immunoglobulins, or can be a single chain antibody. The antibodies can, optionally, include a constant region chosen from a kappa, lambda, alpha, gamma, delta epsilon or a mu constant region gene. A PD-1, LAG-3 and TIGIT marker of the present invention-binding antibody can include a heavy and light chain constant region substantially from a human antibody, as a non-limiting example a human IgG1 constant region or a portion thereof, or from another species, including but not limited to, mouse, rat, dog, cat, goat, sheep, cow, horse, chicken or guinea pig.

In one embodiment, the antibody (or fragment thereof) is a recombinant or modified antibody. In some aspects, the recombinant or modified antibody can be a chimeric, a humanized, a deimmunized, or an in vitro generated antibody. The term “recombinant” or “modified” antibody, as used herein, is intended to include all antibodies that are prepared, expressed, created or isolated by recombinant means, such as antibodies expressed using a recombinant expression vector transfected into a host cell, antibodies isolated from a recombinant, combinatorial antibody library, antibodies isolated from an animal that is transgenic for human immunoglobulin genes or antibodies prepared, expressed, created or isolated by any other means that involves spacing of immunoglobulin gene sequences to other DNA sequences. Such recombinant antibodies include humanized, CDR grafted, chimeric, deimmunized, in vitro generated antibodies, and may optionally include constant regions derived from human germane immunoglobulin sequences.

The VH and VL regions can be further subdivided into regions of hypervariability, termed “complementarity determining regions” (CDR), interspersed with regions that are more conserved, termed “framework regions” (FR). The extent of the framework region and CDRs has been precisely defined (see, Kabat, E. A, et al., Sequences of Proteins of immunological interest, Fifth Edition, U.S. Department of Health and Human Services, NIH Publication No. 91-3242, 1991, and Chothia, C. et al., J. Mol. Biol., 196:901-917, 1987). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two beta-sheets formed of about seven beta-strands, and a conserved disulphide bond (see, A. F. Williams and A. N. Barclay, Ann. Rev Immunol., 6:381-405, 1988). The canonical structures of hypervariable loops of an immunoglobulin variable can be inferred from its sequence, as described in Chothia et al., J Mol. Biol., 227:799-5 817, 1992; Tomlinson et al., J. Biol., 227:776-798, 1992; and Tomlinson et al., EMBO J., 14(18):4628-38, 1995.

As used herein, an “immunoglobulin variable domain sequence” refers to an amino acid sequence which can form the structure of an immunoglobulin variable domain. As a non-limiting example, the sequence may include all or part of the amino acid sequence of a naturally-occurring variable domain. The sequence may omit one, two or more N- or C-terminal amino acids, internal amino acids, may include one or more insertions or additional terminal amino adds, or may include other alterations. In one embodiment, a polypeptide that includes immunoglobulin variable domain sequence can associate with another immunoglobulin variable domain sequence to form a target binding structure (or “antigen binding site”). In some aspects, the antigen binding site may be a structure that interacts with a PD-1, LAG-3 or TIGIT marker of the present invention. In some aspects, the interaction can be binding or inhibiting.

The VH or VL chain of the antibody can further include a or part of a heavy or light chain constant region, to thereby form a heavy or light immunoglobulin chain, respectively. In one embodiment, the antibody can be a tetramer of two heavy immunoglobulin chains and two light immunoglobulin chains, wherein the heavy and light immunoglobulin chains are inter-connected by disulfide bonds. The heavy chain constant region includes three domains, CH1, CH2 and CH3. The light chain constant region includes a CL domain. The variable region of the heavy and light chains contains a binding domain that interacts with an antigen. The constant regions of the antibodies typically mediate the binding of the antibody to a host tissue or factors, including various cells of the immune system (as a non-limiting example, effector cells) and the first component of the classical complement system. The term “antibody” includes intact immunoglobulins of types IgA, IgG, IgE, IgD, IgM (as well as subtypes thereof). In one embodiment the antibody can be an IgA, In another embodiment the antibody can be an IgG. In another embodiment the antibody can be an IgE. In another embodiment the antibody is an IgD. In another embodiment the antibody can be an IgM. The light chains of the immunoglobulin may be of types kappa or lambda. In one embodiment, the antibody can be glycosylated.

One or more regions of an antibody can be human or effectively human. As a non-limiting example, one or more of the variable regions can be human or effectively human. As a non-limiting example, one or more of the CDRs can be human. In some aspects, the human CDRs may be HC CDR1., HC CDR2HC CDR3, LC CDR1, LC CDR2, or LC CDR3. Each of the light chain CDRs can be human. HC CDR3 can be human. One or more of the framework regions can be human. In some aspects, the human framework regions can be FR1, FR2FR3, and FR4 of the HC or LC. In some aspects, all the framework regions are human. In some aspects, the frameworks regions are derived from a human somatic cell. In some aspects, the human somatic cell is a hematopoietic cell that produces immunoglobulins or a non-hematopoietic cell. In some embodiments, the human sequences are germline sequences. In some embodiments, the germline sequences are encoded by a germline nucleic acid.

One or more of the constant regions can be human or effectively human. In some embodiments, at least 70, 75, 80, 85, 90, 92, 95, or 98% of the framework regions (FR1, FR2, and FR3, collectively, or FR1, FR2, FR3, and FR4, collectively) or the entire antibody can be human or effectively human. As a non-limiting example, FR1, FR2, and FR3 collectively can be at least 70, 75, 80, 85, 90, 92, 95, 98, or 99% identical to a human sequence encoded by a human germline V segment of a locus encoding a light or heavy chain sequence.

All or part of an antibody can be encoded by an immunoglobulin gene or a segment thereof. Exemplary immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin light chains can be encoded by a variable region gene at the NH2-terminus and a kappa or lambda constant region gene at the COOH-terminus. Full-immunoglobulin heavy chains can be similarly encoded by a variable region gene and one previously mentioned constant region genes. A light chain refers to any polypeptide that includes a light chain variable domain. A heavy chain refers to any polypeptide that a heavy chain variable domain.

The term “antigen-binding fragment” of a full-length antibody (or simply “antibody portion,” or “fragment”), as used herein, refers to one or more fragments of a full-length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature, 341:544-546, 1989), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See Bird et al., Science, 242:423-426, 1988; and Huston et al., Proc. Natl. Acad. Sci. USA, 85:5879-5883, 1988.

A “humanized” immunoglobulin variable region can be an immunoglobulin variable region that includes sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

As used herein, “binding affinity” refers to the apparent association constant or Ka, Binding affinity may be expressed as the dissociation constant (Kd) which is the reciprocal of the Ka. A target binding agent, such as an antibody may, for example, have a Kd of less than 10⁻⁵, 10⁻⁶, 10⁻⁷ or 10⁻³ M for a particular target molecule. Differences in binding affinity (for specificity or other comparisons) can be at least 1.5, 2, 5, 10, 25, 50, 100, or 1 000-fold.

Binding affinity can be determined by a variety of methods including equilibrium dialysis, equilibrium binding, gel filtration, ELISA, surface plasmon resonance, or spectroscopy (as a non-limiting example, a fluorescence assay). These techniques can be used to measure the concentration of bound and free ligand as a function of ligand (or target) concentration. The concentration of bound ligand (Bound) is related to the concentration of free ligand (Free and the concentration of binding sites for the ligand on the target where (N) is the number of binding sites per target molecule by the following equation:

(Bound)=N((Free)/((1/Ka)+(Free))

Although quantitative measurements of Ka are routine, it is not always necessary to make an exact determination of Ka, though, since sometimes it is sufficient to obtain a qualitative measurement of affinity. Such qualitative measurements of affinity can be determined using a method such as ELISA or FACS analysis, is proportional to Ka, and thus can be used for comparisons, such as determining whether a higher affinity is 2, 5, 10, 20, or 50 fold higher than a reference.

Aptamers.

In some embodiments, the invention also features target protein-binding agents such as aptamers. Aptamers may be nucleic acid aptamers or peptide aptamers. The term “nucleic acid aptamer,” as used herein, refers to a nucleic acid molecule which has a conformation that includes an internal non-duplex nucleic acid structure of at least 5 nucleotides. An aptamer can be a single-stranded nucleic acid molecule which has regions of self-complementarity. “Peptide aptamers” are short peptide sequences presented and conformationally constrained in a robust, inert protein scaffold (Evans et al., Journal of Biology, 7:3, 2008). The three-dimensional conformational constraint of the inserted peptide applied by the protein scaffold readily increases the affinity of the aptamer for the target over that of an unconstrained peptide sequence. Exemplary aptamers include nucleic acid molecules and peptides that bind to PD-1, LAG-3 or TIGIT markers of the present invention. Particular aptamers may be used in place of an antibody in many cases. Other peptides that bind a PD-1, LAG-3 or TIGIT marker of the invention are also included. Peptide-like molecules such as peptoids are further included in the invention. “Peptoids”, or poly-N-substituted glycines, are a class of peptidomimetics whose side chains are appended to the nitrogen atom of the peptide backbone, rather than to the alpha-carbons (as they are in amino acids).

Binding Agent.

The term “binding agent” refers to an agent capable of binding to a PD-1, LAG-3 or TIGIT marker of the present invention under experimental conditions and include, but are not limited to, antibodies and antigen-antibody binding fragments thereof, including but not limited to Fab, Fab′, F(ab′h, scFv or single-domain antibody (sdAb), (also referred to as a nanobody), nucleic acid aptamers, and peptide aptamers.

The PD-1, LAG-3 or TIGIT binding agents have in vitro and in vivo diagnostic utilities. For example, measurement of levels of a PD-1, LAG-3 or TIGIT marker in samples derived from a subject can be used for the diagnosis of HIV. Moreover, the monitoring and quantitation of a PD-1, LAG-3 or TIGIT marker level can be used prognostically to stage the progression of HIV and to evaluate the efficacy of agents used to treat a subject. Such a method can also include contacting a reference sample (such as a “control sample”) with the binding agent, and determining the extent of formation of the complex between the binding agent and the sample relative to the same for the reference sample. A statistically significant change in the formation of the complex in the sample or subject relative to the control sample or subject can be indicative of the presence of a latently infected CDC cell in the sample. The PD-1, LAG-3, or TIGIT markers of the present invention-binding agent can be directly or indirectly labeled with a detectable substance to facilitate detection of the bound or unbound antibody. Suitable detectable substances include various enzymes, prosthetic groups, fluorescent materials, luminescent materials and radioactive materials.

Antiretroviral Therapy:

A term that encompasses any of a number of treatment regimens for subjects who have contracted HIV. Generally, these treatment regimens include a combination of two or more pharmaceutical compositions. Classes of pharmaceutical compositions commonly used in antiretroviral therapy include viral entry inhibitors, nucleoside/nucleotide reverse transcriptase inhibitors, non-nucleoside reverse transcriptase inhibitors, integrase inhibitors, and protease inhibitors.

Binding or Stable Binding:

An association between two substances or molecules, such as the association of an antibody with a peptide, nucleic acid to another nucleic acid, or the association of a protein with another protein or nucleic acid molecule, or the association of a small molecule drug with a protein (such as a tyrosine kinase) or other biological macromolecule. Binding can be detected by any procedure known to one skilled in the art, such as by physical or functional properties. In some aspects, binding can be detected functionally by determining whether binding has an observable effect upon a biosynthetic process such as expression of a gene, DNA replication, transcription, translation, protein activity (including tyrosine kinase activity) and the like.

Biological Sample:

Obtaining a biological sample from a subject includes, but need not be limited to any method of collecting a particular sample known in the art. Obtaining a biological sample from a subject also encompasses receiving a sample that was collected at a different location than where a method is performed; receiving a sample that was collected by a different individual than an individual that performs the method, receiving a sample that was collected at any time period prior to the performance of the method, receiving a sample that was collected using a different instrument than the instrument that performs the method, or any combination of these. Obtaining a biological sample from a subject also encompasses situations in which the collection of the sample and performance of the method are performed at the same location, by the same individual, at the same time, using the same instrument, or any combination of these.

In some embodiments a biological sample can be selected from: a solid tissue sample, blood, plasma, serum, sputum, or urine. In some embodiments, the sample can be a blood sample.

A biological sample encompasses any fraction of a biological sample or any component of a biological sample that may be isolated and/or purified from the biological sample. For example: when cells are isolated from blood or tissue, including specific cell types sorted on the basis of biomarker expression; or when nucleic acid or protein is purified from a fluid or tissue; or when blood is separated into fractions such as plasma, serum, buffy coat PBMC's or other cellular and non-cellular fractions on the basis of centrifugation and/or filtration. A biological sample further encompasses biological samples or fractions or components thereof that have undergone a transformation of mater or any other manipulation.

Biomarker:

Molecular, biological or physical attributes that characterize a physiological or cellular state and that can be objectively measured to detect or define disease progression or predict or quantify therapeutic responses. A biomarker is a characteristic that is objectively measured and evaluated as an indicator of normal biologic processes, pathogenic processes, or pharmacologic responses to a therapeutic intervention. A biomarker may be any molecular structure produced by a cell or organism. A biomarker may be expressed inside any cell or tissue; accessible on the surface of a tissue or cell; structurally inherent to a cell or tissue such as a structural component, secreted by a cell or tissue, produced by the breakdown of a cell or tissue through processes such as necrosis, apoptosis or the like; or any combination of these. A biomarker may be any protein, carbohydrate, fat, nucleic acid, catalytic site, or any combination of these such as an enzyme, glycoprotein, cell membrane, virus, cell, organ, organelle, or any uni- or multimolecular structure or any other such structure now known or yet to be disclosed whether alone or in combination.

A biomarker may be represented by the sequence of a nucleic acid from which it can be derived or any other chemical structure. Examples of such nucleic acids include miRNA, tRNA, siRNA, mRNA, cDNA, or genomic DNA sequences including any complimentary sequences thereof. One example of a biomarker is a DNA coding sequence for a protein comprising one or more mutations that cause amino acid substitutions in the protein sequence.

Contacting:

Placement in direct physical association, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

FACS. (Fluorescent Activated Cell Sorting).

The PD-1., LAG-3, or TIGIT binding agent can be used to label cells or protein. In some aspects, the cells or protein in a biological sample can be a patient sample. The binding protein can also be attached (or attachable) to a fluorescent compound. The cells can then be sorted using fluorescent activated cell sorted. As a non-limiting example, a fluorescent activated cell sorter can be of the type available from Becton Dickinson Immunocytometry Systems, San Jose Calif.; see also U.S. Pat. Nos. 5,627,037; 5,030,002; and 5,137,809), As cells pass through the sorter, a laser beam excites the fluorescent compound while a detector counts cells that pass through and determines whether a fluorescent compound is attached to the cell by detecting fluorescence. The amount of label bound to each cell can be quantified and analyzed to characterize the sample. The sorter can also deflect the cell and separate cells bound by the binding protein from those cells not bound. The separated cells can be cultured and/or characterized.

Inhibit:

To reduce to a measurable extent, for example, to reduce activity of a CD4⁺ T cell latently infected with HIV. Particular outcomes of inhibition include preventing latency from reverting or promoting cell death (including programmed cell death) of the CD4⁺ T cell latently infected with HIV. Preferably, promoting cell death of the CD4⁺ T cell latently infected with HIV does not involve promoting cell death of CD4⁺ T cells lacking latent HIV nucleic acid.

Label:

A detectable compound or composition that can be conjugated directly or indirectly to another molecule to facilitate detection of that molecule. Specific, non-limiting examples of labels include fluorescent tags, enzymatic linkages, and radioactive isotopes. In some embodiments, the label can be a radioactive, fluorescent, colorimeter or enzyme label. In some embodiments, the label can be a second antibody that immunospecifically binds to the anti-PD-1, anti-LAG-3 or anti-TIGIT antibodies. In some examples, a label can be attached to an antibody to facilitate detection of the molecule that the antibody specifically binds.

The terms “labeled antibody” or “tagged antibody”, as used herein, includes antibodies that are labeled by detectable means and include, but are not limited to, antibodies that are fluorescently, electroluminescently enzymatically, radioactively, optical (non-limiting examples are plasmonic resonance, lifetime-based, light scattering), magnetic, or chemiluminescently labeled. Antibodies can also be labeled with a detectable tag, such as c-Myc, HA, VSV-G, HSV, FLAG, V5, or HIS, which can be detected using an antibody specific to the tag, for example, an anti-c-Myc antibody. Antibodies can also be labeled with an enzyme. In some embodiments, the enzyme can be alkaline phosphatase, acid phosphatase, horseradish peroxidase, betagalactosidase or ribonuclease. Antibodies can also be labelled with quantum dots (nanoparticles) which exhibit quantum confinement effects and are therefore subject to stimulated emission such as fluorescence or electroluminescence. Various methods of labeling binding agents are known in the art and may be used. Non-limiting examples of fluorescent labels or tags for labeling the antibodies for use in the methods of invention include QDot605, Brilliant Violet 650, Violet 500, AmCyan, PerCP-eFluor 710, Hydroxycoumarin, Succinimidyl ester, Aminocoumarin, Succinirnidyl ester, Methoxycoumarin, Succinimidyl ester, Cascade Blue, Hydrazide Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate, (FITC), BODIPY-FL, TRITC, X-Rhodamine (XRJTC), Lisamine Rhodamine B, Texas Red, Allophycocyanin (APC), APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, 20 Alexa Fluor 633, Alexa Fluor 647, Alexa Fluor 660, Alexa Fluor 674, Alexa Fluor 680, Alexa Fluor 700, Alexa Fluor 750, Alexa Fluor 790, Cy2, Cy3, Cy38, Cy3.5, Cy5, Cy5.5 or Cy7. A variety of suitable fluorescent species and chromophores are described by Stryer, Science, 162:526, 1968 and Brand, L. et al., Annual Review of Biochemistry, 41:843-868, 1972. The binding proteins can be labeled with fluorescent or chromophore groups by conventional procedures such as those disclosed in U.S. Pat. Nos. 3,940,475, 4,289,747, and 4,376,110. In some embodiments the fluorescent species can be a xanthene dye, which include the fluoresceins and rhodamines. In some embodiments the fluorescent compounds can be a naphthylamine, Once labeled with a fluorophore or chromophore, the binding protein can be used to detect the presence or localization of the PD-1, LAG-3, or TIGIT markers of the present invention in a sample using fluorescent microscopy. In some embodiments the fluorescent microscopy is confocal or deconvolution microscopy, A bioluminescent compound may be used to label the PD-1, LAG-3, or TIGIT markers. The presence of a bioluminescence protein can be determined by detecting the presence of luminescence. Important bioluminescence compounds for purposes of labeling are luciferin, luciferase and aquorin. Other methods of detecting include, but are not limited to, Biacore (surface plasmon resonance), ELISA, histology, and cell-staining.

In some embodiments of the aspects described herein, an agent specific for a PD-1, LAG-3, or TIGIT marker, such as an antibody or antigen-binding fragment thereof, a natural or recombinant ligand, a small molecule, or a modifying moiety, is directly labeled with a tag to facilitate the detection of the modification. The terms “label” or “tag”, as used herein, refer to a composition capable of producing a detectable signal indicative of the presence of a target, such as, the presence of a specific modification in a biological sample. Suitable labels include fluorescent molecules, radioisotopes, nucleotide chromophores, enzymes, substrates, chemiluminescent moieties, magnetic particles, bioluminescent moieties, peptide tags (c-Myc, HA, VSV-G, HSV, FLAG, V5 or HIS) and the like. As such, a label is any composition detectable by spectroscopic, photochemical, biochemical, immunochemical, electrical, optical or chemical means needed for the methods to identify the PD-1, LAG-3, or TIGIT markers. In some embodiments of the aspects described herein, the modification moiety itself may be labeled directly. As a non-limiting example, one can use a radioactive label or a fluorescent label so that the protein modification can be read directly (or in combination with other modifications) without the use of antibodies.

Latent HIV Nucleic Acid:

Any form of HIV that is present within the CD4⁺ T cell pool of an HIV infected subject, particularly an HIV infected subject who is currently undergoing antiretroviral therapy. Latent HIV nucleic acid can take many forms including HIV DNA that has integrated in the genome and unspliced HIV RNA.

Polypeptide:

Any chain of amino acids, regardless of length or posttranslational modification (such as glycosylation, methylation, ubiquitination, phosphorylation, or the like). “Polypeptide” is used interchangeably with “protein,” and is used to refer to a polymer of amino acid residues. A “residue” refers to an amino acid or amino acid mimetic incorporated in a polypeptide by an amide bond or amide bond mimetic.

Sample:

A sample, such as a biological sample, is a sample obtained from a human or animal subject, such as a sample comprising CD4⁺ T cells. Samples include, but are not limited to, cells, tissues, and bodily fluids, including tissues that are, for example, unfixed, frozen, fixed in formalin and/or embedded in paraffin.

Sequence Identity/Similarity:

The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl, Math., 2:482, 1981; Needleman & Wunsch, J. Mol. Biol., 48:443, 1970; Pearson & Lipman, Proc, Natl. Acad. Sci. USA, 85:2444, 1988; Higgins & Sharp, Gene, 73:237-44, 1988; Higgins & Sharp, CABIOS, 5:151-3, 1989; Corpet et al., Nuc. Acids Res., 16:10881-90, 1988; Huang et al., Computer Appls. in the Biosciences, 8, 155-65, 1992; and Pearson et al., Meth. Mol. Bio., 24:307-31, 1994. Altschul et al., J. Mol. Biol., 215:403-10, 1990, presents a detailed consideration of sequence alignment methods and homology calculations.

The NCBI Basic Local Alignment Search Tool (BLAST) (Altschul et al., J. Mol, Biol., 215:403-10, 1990) is available from several sources, including the National Center for Biological Information (NCBI, National Library of Medicine, Building 38A, Room 8N805, Bethesda, Md. 20894) and on the Internet, for use in connection with sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino 5 acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBI Basic BLAST 2.0, gapped blastp with databases such as the nr or swissprot database. Queries searched with the blastn program are filtered with DUST (Hancock and Armstrong, Comput, Appl. Biosci., 10:67-70, 1994). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the BLAST2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code, Changes in a nucleic acid sequence can be made using this degeneracy to produce multiple nucleic acid molecules that all encode substantially the same protein. An alternative and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Subject:

A living multicellular vertebrate organism, a category that includes, for example, mammals and birds. A “mammal” includes both human and non-human mammals, such as mice. In some examples, a subject is a patient, such as a patient that is HIV-positive including a patient that is HIV-positive and undergoing antiretroviral therapy (ART).

Test Compound:

A candidate molecule that is tested for its ability to inhibit a CD4⁺ T cell latently infected with HIV, preferably with minimal effects on normal CD4⁺ T cells. The test compound can include any small organic molecule, or a biological entity. As non-limiting examples, the test compound can be a protein (such as an antibody or a peptide), a sugar, a nucleic acid (such as an antisense oligonucleotide, a ribozyme, or RNAi molecule) or a lipid. The test compound may be isolated, or may be part of a mixture (for example two or more test compounds). The test compound or mixture of test compounds may also include additional components, such as diluents, solvents, pharmaceutically acceptable carriers, or other compounds. Test compounds can also include positive or negative controls known to inhibit or kill CD4⁺ T cells.

Treating.

As used herein, the term “treating” refers its meaning as known in the art, and to both therapeutic treatment and prophylactic, or preventative, measures, or administering an agent suspected of having therapeutic potential. The term includes preventative (as a non-limiting example, prophylactic) and palliative treatment. As used herein, the term “treatment” also includes symptomatic therapy to lessen, alleviate, or mask the symptoms of the disease or disorder, as well as therapy for preventing, lowering, stopping, or reversing the progression of severity of the condition or symptoms being treated. As such, the term “treatment” includes both medical therapeutic treatment of an established condition or symptoms and/or prophylactic administration, as appropriate.

Methods of Identifying Cells with Latent HIV Nucleic Add

Disclosed herein are methods of identifying a cell from a subject as having latent HIV nucleic acid. The methods involve obtaining a biological sample from the subject. The biological sample is contacted with one or more labeled antibodies. The labeled antibodies are specific for PD-1, LAG-3, or TIGIT markers and form a complex with said markers such that any cell in the sample that expresses PD-1, LAG-3, or TIGIT will be bound by the labeled antibody. The labels conjugated to said antibodies can be detected by any appropriate method. Detection of the complex signifies that the cell includes latent HIV nucleic acid. The use of the combination of the labelled antibodies to PD-1, LAG-3, or TIGIT markers to detect CDC cells with latent HIV nucleic acid is a surprising effective diagnostic to distinguish patient prognosis for HIV.

LAG-3 is also known as CD223, and can be detected with a commercially available labelled antibody such as Human LAG-3 FITC-conjugated Antibody from polyclonal goat IgG (R&D Systems #FAB2319F). Such a labelled antibody is antigen purified with a specificity of less than 1% cross-reactivity with mouse LAG-3. TIGIT is also known as Tg with ITIM domains, and is also known as VSTM3 or WUCAM, and can be detected with a commercially available labelled monoclonal antibody such as TIGIT-PerCP eFluor 710 (eBioscience #46-9500, clone MBSA43). PD-1 is also known as CD279 and can be detected with a commercially available monoclonal labelled antibody such as Anti-human CD279 (PD-1) APC (eBioscience 417-9969-4).

The sample may be any sample that includes CD4⁺ T cells, including blood, lymph, and/or a lymph node biopsy such as a needle biopsy. Prior to contacting the biological sample with the labeled antibody, the biological sample may be further processed, for example by isolating mononuclear cells by centrifugation, isolating CDC cells through cell sorting (fluorescent or magnetic) or by any other processing method known in the art.

The labeled antibodies can be conjugated to a fluorescent, chemiluminescent enzymatic, magnetic, metallic, chemical, radioactive, or other label that signifies and/or locates the presence of specifically bound antibody. In some aspects, the conjugation can be achieved by the appropriate chemical linkage (see Hermanson, G. T., Bioconjugate Techniques, 3^{rd} Ed., Academic Press, 2013). In some aspects, the labeled antibodies can be conjugated to a fluorescent compound such as fluorescein isothiocyanate, rhodamine, phycoerythrin, phycocyanin, allophycocyanin, a-phthaldehyde and fluorescamine or any other appropriate fluorescent compound known in the art.

In some aspects of the invention, the label helps facilitate isolating (interchangeably described as sorting or purifying) the cells, for example through the use of fluorescent activated cell sorting (FAN, sorting using magnetic beads, or through binding to an affinity column.

The complex formation between a PD-1, LAG-3, or TIGIT-binding agent and a PD-1, LAG-3, or TIGIT marker of the present invention can be detected by measuring or visualizing either the binding agent bound to the PD-1, LAG-3, or TIGIT marker or unbound binding agent. Assays (immunoassays) of the invention include competitive and noncompetitive (“sandwich”) assays. Immunoassays of the invention include but are not limited to assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement fixation assays, immunoradiometric assays, fluorescent immunoassays, protein A immunoassays, flow cytometry or tissue immunohistochemistry. Immunohistochemistry can be performed using a PD-1, LAG-3, or TIGIT-binding agent (as non-limiting examples: an antibody, antigen binding fragment thereof, or aptamer). In some aspects, the antibody can be synthesized with a label (such as a purification or epitope tag), or can be detectably labeled by conjugating a label or label-binding group. In some aspects, a chelator can be attached to the antibody. The antibody can then be contacted to a histological preparation, as a non-limiting example, a fixed section of tissue that is on a microscope slide. After an incubation for binding, the preparation can be washed to remove unbound antibody. The preparation can then be analyzed using microscopy to identify if the antibody is bound to the preparation. The method can be used to evaluate a cell or tissue sample. The antibody (or other polypeptide or peptide) can be unlabeled at the time of binding. After binding and washing, the antibody can be labelled in order to render it detectable.

The detection of labeled antibodies in biological samples can also be used to monitor the efficacy of potential anti-HIV agents during treatment. In some aspects, the levels of labeled antibodies to PD-1LAG-3, or TIGIT can be determined before and during treatment.

The efficacy of the treatment agent can be followed by comparing the expression of PD-1, LAG-3, or TIGIT or any mixture thereof, throughout the treatment. Agents exhibiting efficacy are those which decrease the level of a PD-1, LAG-3, or TIGIT marker as treatment with the agent progresses.

In some examples, the sample is contacted with a second labeled antibody including a second labeled antibody that specifically binds CD4. For CD4, the label on the second antibody is distinguishable from the label on the first antibody so as to differentiate CD4⁺ cells latently infected with HIV from CD4⁺ cells that do not. In some aspects, additional antibodies may be used that bind to another 1CB. As a non-limiting example, a first antibody that binds PD-1 and a second antibody that binds LAG-3 can be used; or a first antibody that binds PD-1 and a second antibody that binds TIGIT can be used; or a first antibody that binds LAG-3 and a second antibody that binds TIGIT can be used; or a first antibody that binds PD-1, a second antibody that binds LAG-3, and a third antibody that binds TIGIT can be used. Any of these combinations may be used with an antibody that binds CD4. Any of these combinations can be used with an antibody that binds another ICB such as BTLA, 284, CTLA4, TIM-3, or CD160. Any of these combinations can be used with any other antibody with any other specificity.

In some aspects, the method involves purifying the cells, preferably through a method facilitated by the label conjugated to the antibody. Such purification methods can include fluorescence activated cell sorting or magnetic sorting.

This invention relates to CD4⁺ T cells latently infected with HIV which are isolated by the disclosed methods. These CD4⁺ T cells may be used for any of a number of purposes including methods of screening for inhibitors of CD4⁺ T cells latently infected with HIV. Such screening methods involve contacting CD4⁺ T cells latently infected with HIV with a test compound and assessing the ability of the test compound to inhibit the CD4⁺ T cells latently infected with HIV. In some aspects, the test compound is also contacted with control CD4⁺ T cells that lack latent HIV nucleic acid. In some embodiments, the control CD4⁺ T cells are from a healthy donor. In some embodiments, the test compound is an antibody specific for PD-1, LAG-3 or TIGIT markers.

In one aspect the invention provides for a method for the diagnosis of HIV in a subject comprising: obtaining a biological sample from the subject, the biological sample comprising CD4⁺ T cells; contacting the sample with a first labeled antibody comprising a first label, wherein the first labeled antibody specifically binds PD-1, LAG-3, or TIGIT; forming a complex between the first labeled antibody to PD-1, LAG-3, or TIGIT with PD-1, LAG-3, or TIGIT; detecting the first label on the surface of a CD4⁺ cell that has bound the labeled antibody; wherein detection of the first label indicates that the cell is a latently infected CD4⁺ T cell.

In some aspects, the present invention relates to diagnostic and prognostic methods for diseases such as HIV based on detection of PD-1, LAG-3 or TIGIT markers in a subject. The method may be validated by the use of a biological sample from a subject with HIV and from age and gender matched controls, without HIV. A biological sample which may contain CDC cells latently infected with HIV, such as urine, blood, serum or plasma, is obtained from a subject having or suspected of having HIV or suspected of being predisposed to developing HIV. A corresponding body fluid may be obtained from a subject that does not have HIV as a control.

In some aspects, the method of detecting the first label can be performed using flow cytometry. In some aspects, the method can comprise contacting the sample with a second labeled antibody, wherein the second labeled antibody binds CD4. In some aspects, the method can comprise isolating the CD4⁺ T cells that have bound the first antibody. In some aspects, the isolation of the CD4⁺ T cells can be achieved with fluorescently activated cell sorting or magnetic sorting.

The present invention relates to a population of isolated CD4⁺ T cells. In some aspects, said cells can be isolated using fluorescently activated cell sorting or magnetic sorting. In some aspects, said cells can be isolated from a subject undergoing antiretroviral therapy.

The present invention relates to a method of selecting a compound that inhibits a latently infected CD4⁺ T cell by contacting the isolated CD4⁺ T cell with a test compound and assessing whether or not the test compound inhibited said CD4⁺ T cell from expressing PD-1, LAG-3, or TIGIT markers. In some aspects, the isolated CD4⁺ T cell can be isolated by fluorescently activated cell sorting or magnetic sorting. In some aspects, the method can involve contacting a control CD4⁺ T cell with the test compound wherein the control CD4⁺ T cell is known not to comprise latent HIV nucleic acid. In some aspects, the control CD4⁺ T cell is a CD4⁺ T cell from an HIV-negative subject. In some aspects, the test compound can comprise an antibody specific for PD-1, LAG-3, or TIGIT.

The preceding merely illustrates the principles of the invention. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art, and are to be construed as being without limitation to such specifically recited examples and conditions.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

### EXAMPLES

The following examples are illustrative of disclosed methods. In light of this disclosure, those of skill in the art will recognize that variations of these examples and other examples of the disclosed method would be possible without undue experimentation.

### Example 1

### Methods

Expression of PD-1, CTLA-4, LAG-3, TIGIT, TIM-3, BTLA, 2B4 and CD160 were measured by flow cytometry on peripheral blood mononuclear cells (PBMCs) from 48 subjects on ART for >3 years with HIV viral load <50 copies/ml and with a CD4 count >350 cells/μL. The frequencies of CD4 T cells harboring integrated HIV DNA, total HIV DNA, 2-LTR circles and cell associated unspliced (CA-US) HIV RNA were determined by qPCR. Integrated HIV DNA was also measured in sorted memory CD4 T cell subsets expressing some of these ICBs. More specifically, the impact of PD-1 engagement on HIV reactivation was assessed in CD4⁺ T cells isolated from virally suppressed subjects using beads coated with anti-CD3, anti-CD28, PD-L1 or the appropriate control.

Antibodies and other reagents used: CD3-Alexa 700 (Becton Dickinson #557943, clone UCHTI), CD4-Qdot605 (Invitrogen/Life Technologies/Thermo Fisher #Q10008, clone S3.5), CD8-PacificBlue (Becton Dickinson #558207, clone RPA-T8), CD45RA-APC H7 (Becton Dickinson #560674, clone Hl 00), CD27-BV650 (Biolegend #302827, 0323), CCR7-PE Cy7 (Becton Dickinson #557648, clone 3012), CD14-V500 (Becton Dickinson #561391, clone M5E2), CD19-Amcyan (Becton Dickinson #339190, clone SJ25CI), Live/Dead Viability Aqua Dead Cell Stain (Invitrogen #L34957), POI (CD279)-Alexa647 (Becton Dickinson #560838, clone EH12.1), LAG3 (CD223)-FITC (R&D systems #FAB2319F) TIGIT-PerCP eFluor710 (eBioscience #46-9500, clone MBSA43).

### Example 2

### PD-1, LAG-3 and TIGIT in CD4⁺ T cells

Absolute CD4⁺ T cell counts were negatively correlated with the expression of PD-1, LAG-3 and TIGIT in CD4 T-cells (r=−0.53 p<0.0001, r=−0.51 p=0.0002, r=−0.40 p=0.005, respectively). Interestingly, the frequency of CD4 T cells harboring integrated HIV DNA was positively correlated with the expression of these markers (r=0.29 p=0.06, r=0.31 p=0.04 and r=0.46 p=0.002, for PD-1, LAG-3 and TIGIT respectively). With the exception of TIGIT with 2-LTR circles (r=0.39 p=0.009), no statistically significant associations were found between these markers and total HIV DNA, 2-LTR circles or CA-US HIV RNA. Memory CD4 T cells expressing high levels of PD-1 or LAG-3 were enriched for HIV integrated DNA when compared to their negative counterparts. Engagement of PD-1 with its ligand PD-L1 inhibited viral production induced by TCR stimulation in latently infected cells (mean=94.8% of inhibition), suggesting a functional role for this receptor in the maintenance of HIV latency.

PD-1, LAG-3 and TIGIT have been identified as markers associated with incomplete CD4⁺ T cell restoration and HIV persistence during ART. The data further demonstrate that PD-1 and LAG-3 identify cells carrying integrated HIV DNA in virally suppressed subjects.

### Example 3

### ICBs Show Differential Expression in CD4 T Cells from Virally Suppressed HIV Infected Subjects

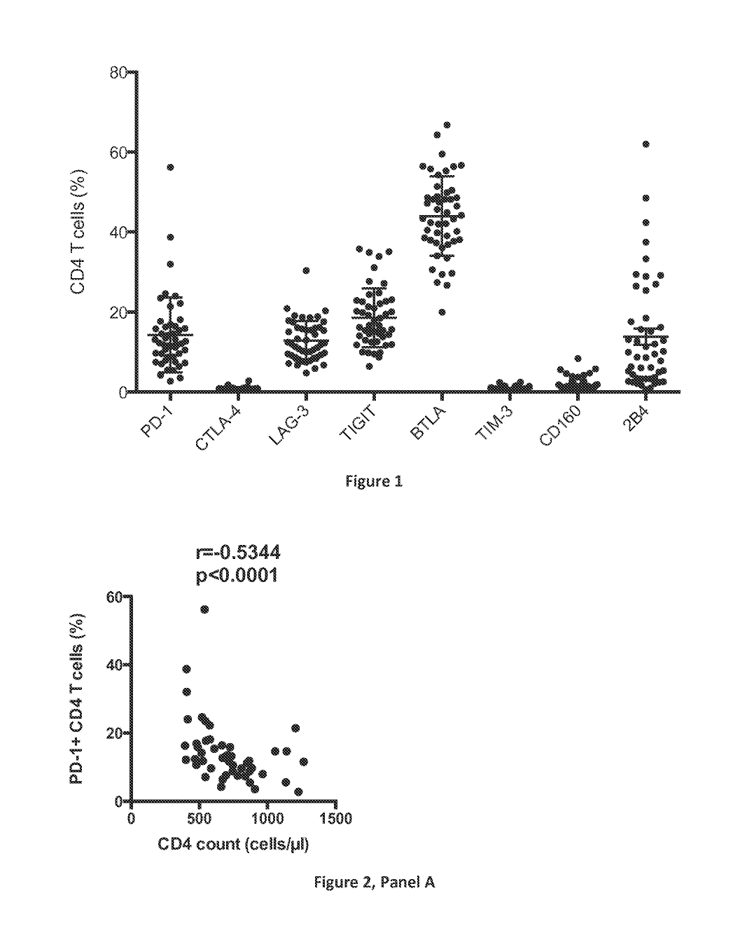

In FIG. 1, the levels of expression of eight selected ICBs (PD-1, LAG-3, TIGIT, CTLA-4, BTLA, CD160, 284, TIM-3) were measured by multiparametric flow cytometry on CD4⁺ T cells from 48 HIV infected subjects on suppressive ART for at least 3 years.

### Example 4

### The Percentage of CD4⁺ T Cells Expressing PD-1, LAG-3, and TIGIT are Associated with Incomplete CD4 Restoration During ART

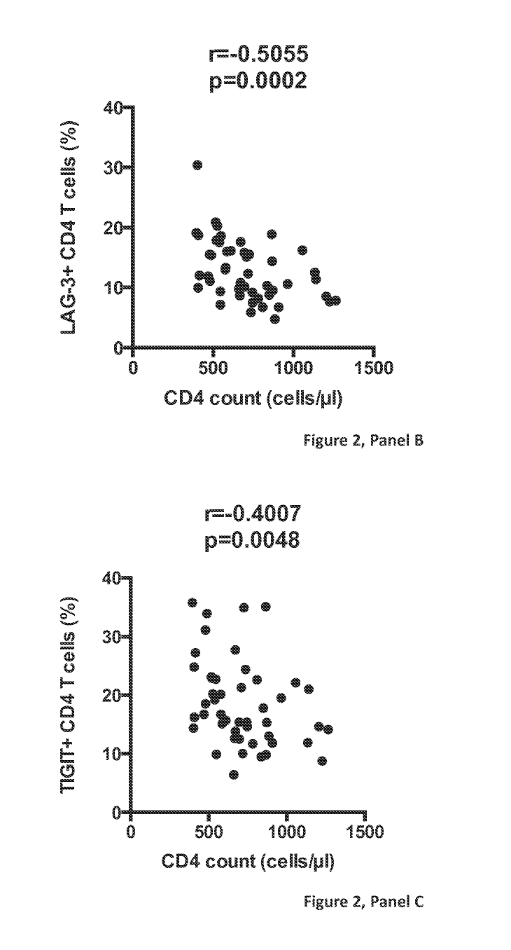

FIGS. 2A-C shows the association between the frequency of CD4 T cells expressing PD-1, LAG-3, and TIGIT and the CD4 count was addressed using a Spearman's rank correlation. Absolute CD4 T cell counts were negatively correlated with the expression of PD-1, LAG-3 and TIGIT in CD4 T-cells (r=−0.53 p<0.0001, r=−0.51 p=0.0002, r=−0.40 p=0.005, respectively).

### Example 5

### Integrated, Total, 2-LTR HIV DNA and UnSpliced HIV RNA are Detected in CD4 T Cells from Virally Suppressed HIV Infected Subjects

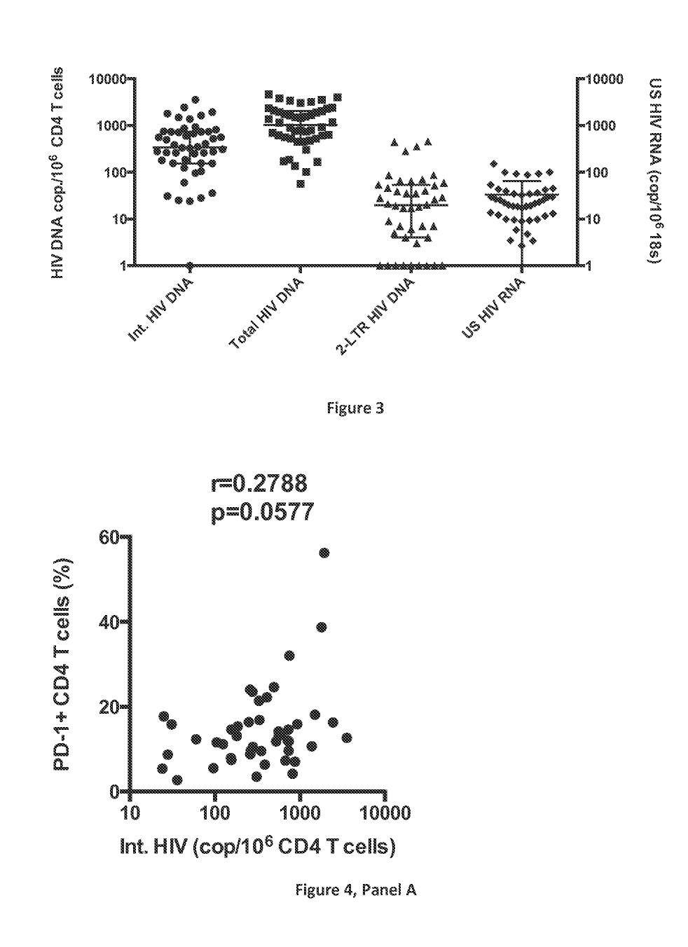

FIG. 3 shows the results of quantifications of HIV DNA forms (total, integrated and 2-LTR circles) performed by real time nested PCR on total CD4 T cells lysed in proteinase K, as described in Chomont N et al., Nat Med, 15, 893-900, 2009, incorporated by reference herein. Cell associated US HIV RNA was quantified on extracted RNA from CD4 T cells and normalized to 185 RNA. Integrated, total, 2-LTR HIV DNA and US HIV RNA were detected in 98%, 100%, 80% and 100% of the samples, respectively.

### Example 6

### The Frequencies of CD4 T Cells Expressing PD-1, LAG-3, TIGIT are Associated with HIV Persistence During ART

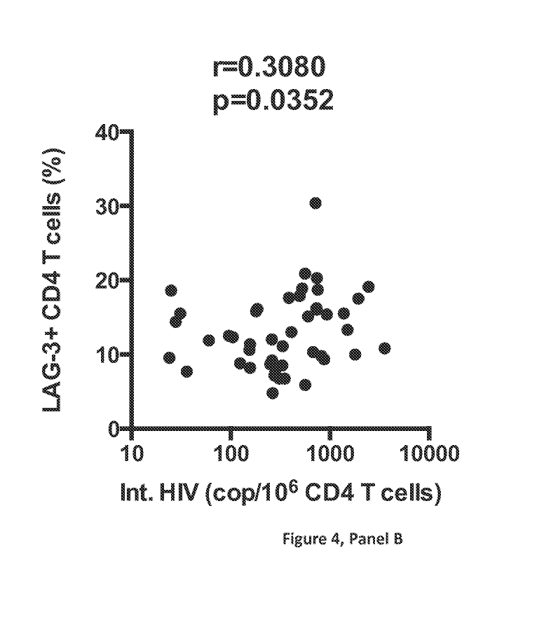

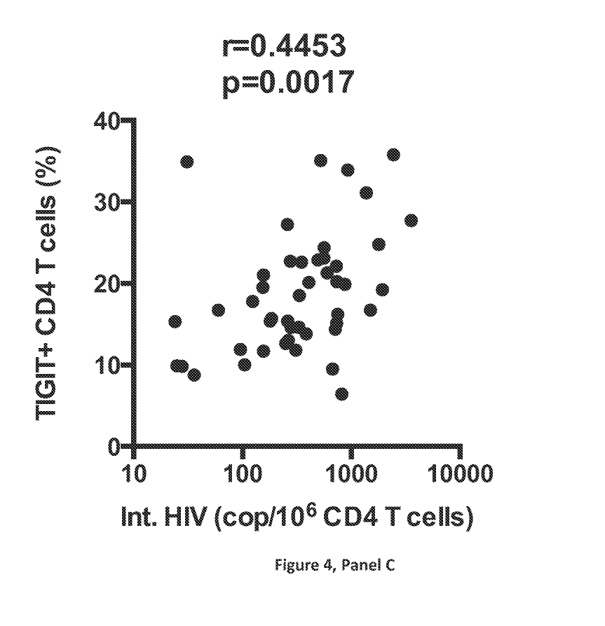

FIGS. 4A-C show that the frequency of CD4 T cells harboring integrated HIV DNA was positively correlated with the expression of PD-1, LAG-3 and TIGIT (r=0.29 p=0.06, r=0.31 p=0.04 and r=0.46 p=0.002). Correlations were determined with Spearman test.

SEQUENCE LISTING

```
SEQ. ID NO: 1 is an amino acid sequence of
human PD-1
    1 mqipqapwpv vwavlqlgwr pgwfldspdr pwnpptfspa
        llvvtegdna tftcsfsnts
  61 esfvlnwyrm spsnqtdkla afpedrsqpg qdcrfrvtql
        pngrdfhmsv vrarrndsgt
121 ylcgaislap kaqikeslra elrvterrae vptahpspsp
        rpagqfqtlv vgvvggllgs
181 lvllvwvlav icsraargti garrtgqplk edpsavpvfs
        vdygeldfqw rektpeppvp
241 cvpeqteyat ivfpsgmgts sparrgsadg prsaqplrpe
        dghcswpl
SEQ ID NO: 2 is an amino acid sequence of
human LAG-3
    1 mweaqflgll flgplwvapv kplqpgaevp vvwagegapa
        qlpcsptipl qdlsllrrag
  61 vtwqhqpdsg ppaaapghpl apgphpaaps swgprprryt
        vlsvgpgglr sgrlpiqprv
121 qldergrqrg dfslwarpar radageyraa vhlrdralsc
        rlrlrlgqas mtasppgslr
181 asdwvilncs fsrpdrpasv hwfrnrgqgr vpvresphhh
        laesflflpq vspmdsgpwg
241 ciltyrdgfn vsimynltvl glepptpltv yagagsrval
        pcrlpagvgt rsfltakwtp
301 paggpdllvt gdngdftlrl edvsqaqagt ytchihlqeq
        qlnatvtlai itvtpksfgs
361 pgslgkllce vtpvsggerf vwssldtpsq rsfsgpwlea
        qeaqllsqpw qcglyqgerl
421 lgaavyftel sspgaqrsgr apgalpaghl llfltlgvls
        llllvtgafg fhlwrrqwrp
481 rrfsaleqgi hppqaqskie eleqepepep epepepepep
        epeql
SEQ ID NO: 3 is an amino acid sequence of
        human TIGIT
    1 mrwcllliwa qglrqaplas gmmtgtiett gnisaekggs
        iilqchlsst taqvtqvnwe
  61 qqdqilaicn adlgwhisps fkdrvapgpg lgltlgsltv
        ndtgeyfciy htypdgtytg
121 riflevless vaehgarfgi pllgamaatl vvictavivv
        valtrkkkal rihsvegdlr
181 rksagqeews psapsppgsc vqaeaapagl cgeqrgedca
        elhdyfnvls yrslgncsff
241 tetg
SEQ ID NO: 4 is an amino acid sequence of
human CD4
    1 mnrgvpfrhl llvlqlallp aatqgkkvvl gkkgdtvelt
        ctasqkksiq fhwknsnqik
  61 ilgnqgsflt kgpsklndra dsrrslwdqg nfpliiknlk
        iedsdtyice vedgkeevql
121 lvfgltansd thllqgqslt ltlesppgss psvqcrsprg
        kniqggktls vsqlelqdsg
181 twtctvlqnq kkvefkidiv vlafqkassi vykkegeqve
        fsfplaftve kltgsgelww
241 qaerasssks witfdlknke vsvkrvtqdp klqmgkklpl
        hltlpqalpq yagsghltla
301 leaktgklhq evnlvvmrat qlqknltcev wgptspklml
        slklenkeak vskrekavwv
361 lnpeagmwqc llsdsgqvll esnikvlptw stpvqpmali
        vlggvaglll figlgiffcv
421 rcrhrrrqae rmsgikrlls ekktcqcphr fqktcspi
```

What is claimed is:

1. A method of identifying a latently infected CD4⁺ T cell from a subject, the method comprising:
obtaining a biological sample from the subject, the biological sample comprising CD4⁺ T cells;
contacting the sample with a first labeled antibody comprising a first label, wherein the first labeled antibody specifically binds PD-1, LAG-3, or TIGIT;
forming a complex between the first labeled antibody to PD-1, LAG-3, or TIGIT with PD-1, LAG-3, or TIGIT;
detecting the complex by detecting the first label on the surface of a CD4⁺ cell that has bound the labeled antibody in the complex;
wherein detection of the first label indicates that the cell is a latently infected CD4⁺ T cell.

2. The method of claim 1 wherein detecting the first label is performed using flow cytometry.

3. The method of claim 1 further comprising contacting the sample with a second labeled antibody, wherein the second labeled antibody binds CD4.

4. The method of claim 1 further comprising isolating the CD4⁺ T cells that have bound the first antibody.

5. The method of claim 4 wherein isolating further comprises fluorescently activated cell sorting or magnetic sorting.

6. A population of CD4⁺ T cells isolated by the method of claim 4 or 5.

7. The population of CD4⁺ T cells from claim 6 wherein the population is isolated from a subject undergoing antiretroviral therapy.

8. A method of selecting a compound that inhibits a latently infected CD4⁺ T cell, the method comprising:
contacting the CD4⁺ T cell of claim 6 or 7 with a test compound;
assessing whether or not the test compound inhibited a CD4⁺ T cell of claims 6-7.

9. The method of claim 8 further comprising contacting a control CD4⁺ T cell with the test compound wherein the control CD4⁺ T cell is known not to comprise latent HIV nucleic acid.

10. The method of claim 9 wherein the control CD4⁺ T cell is a CD4⁺ T cell from an HIV-negative subject.

11. The method of claim 9 wherein the test compound comprises an antibody specific for PD-1, LAG-3, or TIGIT.
